(19) United States
(12) Patent Application Publication
(10) Pub. No.: US 2006/0178565 A1
(43) Pub. Date: Aug. 10, 2006

(54) Electronic endoscope system

(75) Inventors: Go Matsui, Saitama (JP); Takayuki Enomoto, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/346,264

(22) Filed: Feb. 3, 2006

(30) Foreign Application Priority Data
Feb. 7, 2005 (JP) P2005 - 030686

(51) Int. Cl. A61B 1/06; A61B 6/00

(52) U.S. Cl. 600/160; 600/476; 600/109

(57) ABSTRACT

An electronic endoscope system has a light source unit that is capable of selectively emitting normal-light and exciting-light, and a video-scope with an image sensor, which has an exciting-light eliminating or cutoff filter, and an image processor that processes image signals read from the image sensor. The image signal processor processes the image signals so as to compensate for a change of at least one of luminance and color in an observed image that occurs due to light blocked in accordance with the spectrum transmittance characteristics of the exciting-light eliminating filter.

### BACKGROUND OF THE INVENTION

1. Field of the Invention

The present invention relates to an electronic endoscope system that is capable of displaying auto-fluorescent image for observing or diagnosing a lesion such as a cancer. Especially, it relates to a signal process when a video-scope, adapted to display auto-fluorescent image, is used.

2. Description of the Related Art

In an electronic endoscope system with an auto-fluorescent observation function, light, having a wavelength in the ultraviolet range or in that vicinity (hereinafter, called “exciting-light”), is emitted to the epithelium of an organ such as the lungs. Tissue in the epithelial layer has a fluorescent substance, which emits fluorescent light when the exciting-light is illuminated there on. A subject image is formed on an image sensor provided on a tip portion of a video-scope, due to the fluorescent light passing through an objective lens, so that an image based on fluorescent light (hereinafter, called an “auto-fluorescent image”) is displayed on a monitor. Since the amount of auto-fluorescent light that a lesion or abnormal tissue emits is weak compared to the normal tissue, luminance of the lesion or the area adjacent to the lesion in an auto-fluorescent image is relatively small, thus the lesion can be easily detected compared to a normal image obtained by a white light emitted from such as a xenon lamp.

Since the exciting-light that is reflected on the epithelial layer and that is directed toward the image sensor becomes an obstruction when forming the auto-fluorescent image, a filter for blocking or cutting off light having a wavelength corresponding to the exciting-light, is provided at the front of the image sensor.

### SUMMARY OF THE INVENTION

An object of the present invention is to provide an electronic endoscope system that is capable of displaying an observed image with an adequate luminance and color in a condition where a video-scope with a filter for blocking the exciting-light is used. An electronic endoscope system according to the present invention has a light source unit that is capable of selectively emitting normal-light and exciting-light. The normal-light is utilized for normal-observation, namely, for displaying a normal color observed image. The normal-light is generally white light, and the spectrum distribution is generally uniform over the wavelength of visible-light. Exciting-light is the light used for emitting auto-fluorescent light from tissue in an epithelial layer, and has a specific wavelength. The wavelength of the exciting-light is basically in the range of ultraviolet light, however, there are other types of exciting-light, which have a given wavelength included in the wavelength of the visible-light. The exciting-light makes the tissue emit auto-fluorescent light, by which an auto-fluorescent image is formed. For example, the normal-light or fluorescent light is selectively emitted in accordance with an operation of a button. Further, when generating composite signals, wherein an auto-fluorescent image is superimposed on a normal image, the light source unit alternately emits the normal-light and the exciting-light while synchronizing with the signal-reading time-intervals.

In the electronic endoscope system, a plurality of video-scopes is selectively used and connected to an endoscope component, such as a video-processor with a light source unit or an exclusive light-source unit. The video-scope with an image sensor has an exciting-light eliminating or cut off filter. The exciting-light eliminating filter is provided in front of the image sensor, and blocks or cuts off the exciting-light. An image signal processor provided in electronic endoscope system processes image signals read from the image sensor, to generate video signals. To enable one video-processor or the light source unit to be adaptable various types video-scopes, for example, a video-scope detector that detects the spectrum transmittance characteristics of the exciting-light eliminating filter provided in a connected video-scope, is provided. The light source unit emits exciting-light depending upon the spectrum transmittance characteristics.

According to the present invention, the image signal processor processes the image signals so as to compensate for a change of at least one of luminance and color in an observed image. This change occurs due to light blocked in accordance with the spectrum transmittance characteristics of the exciting-light eliminating filter. Each exciting-light eliminating filter has particular spectrum transmittance characteristics, one eliminating filter cuts off light having a wavelength in the range of ultraviolet light, other filters cut off light having a wavelength in the visible-light. The image processor processes the image signals, not to cause a change of the observed image with respect to luminance or color, or both luminance and color. The image signals are automatically corrected so that an observed image with adequate color and luminance quality is displayed regardless of the type of video-scope, and the spectrum transmittance characteristics of the filter. For example, the image signal processor can be provided in the video-scope.

To process the image signals using an easy construction, for example, the image signal processor has a signal processing circuit, and a signal controller. The signal processing circuit processes the image signals by using coefficients, such as a matrix-operation for generating Red (R), green (G), Blue (B) signals or luminance and color difference signals (Y, Cb, Cr). The signal controller sets the coefficients to compensation coefficients that compensate for the change. For example, the signal controller sets normal-light coefficients corresponding to the normal-light as the compensation coefficients, and sets exciting-light coefficients corresponding to the fluorescent light as the compensation coefficients. The signal processing circuit processes the image signals by using the normal-light coefficients when the light source unit emits normal-light, and processes the image signals by using the exciting-light coefficients when the light source unit emits exciting-light.

To perform compensation process by utilizing a data process, a first memory that stores the compensation coefficients as data, and a second memory that is provided in the signal processing circuit and stores the compensation coefficients as data, are provided. The signal controller reads the compensation coefficients from the first memory and writes the compensation coefficients in the second memory. The signal processing circuit processes the image signals in accordance with the written compensation coefficients.

A video-scope that does not adapt to the auto-fluorescent observed image, namely, that does not have an exciting-light eliminating filer, is connectable to the video-processor, or the light source unit. To prohibit the radiation of the exciting-light in a case where such a video-scope is connected, a video-scope type detector and a light source controller are provided. The video-scope type controller detects whether a connected video-scope is adaptable for the exciting-light. The light source controller prevents the light source unit from emitting the exciting-light when the connected video-scope is not adaptable to the exciting-light.

### BRIEF DESCRIPTION OF THE DRAWINGS

The present invention will be better understood from the description of the preferred embodiment of the invention set forth below together with the accompanying drawings, in which:

FIG. 1 is a block diagram of an electronic endoscope system according to the present embodiment;

FIG. 2 is a block diagram of the video-scope;

FIG. 3 is a block diagram of the video-processor;

FIG. 4 is a block diagram of the light source unit;

FIG. 5 is a front view of a rotary shutter;

FIG. 6 is a block diagram of the video-scope;

FIG. 7 is a view showing data associated with the signal process;

FIG. 8 is a flow chart of initial setting process performed by the system control circuit; and

FIG. 9 is a flowchart of the main routine performed by the system control circuit.

### DESCRIPTION OF THE PREFERRED EMBODIMENTS

Hereinafter, the preferred embodiment of the present invention is described with reference to the attached drawings.

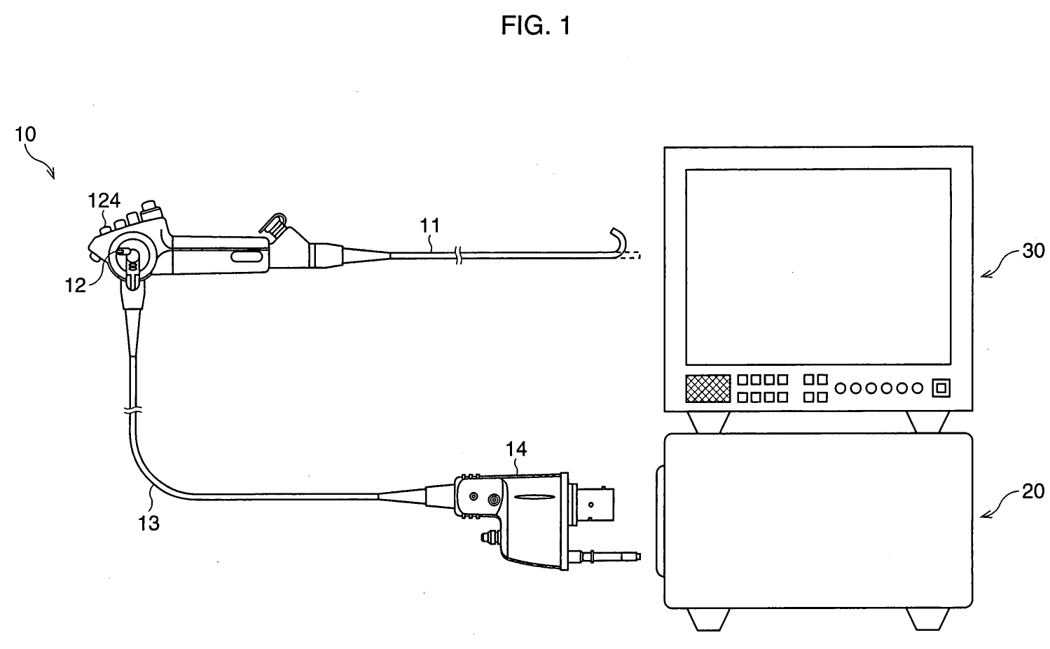

FIG. 1 is a block diagram of an electronic endoscope system according to the present embodiment.

The electronic endoscope system has a video-scope 10, a processor 20, and a monitor 30. The video scope 10 is constructed of an inserted portion 11, an operated portion 12, a cable 13, and a connected portion 14.

An observation change button 124 for changing an observed image is provided on the operated portion 12. In this embodiment, when displaying an observed-image, one of a normal-observation mode, a fluorescent-observation mode, and a special-observation mode is selectively set by an operator. When the connected portion 14 is connected to the video-processor 20, electric power is supplied from the video-processor 20 to the video-scope 10.

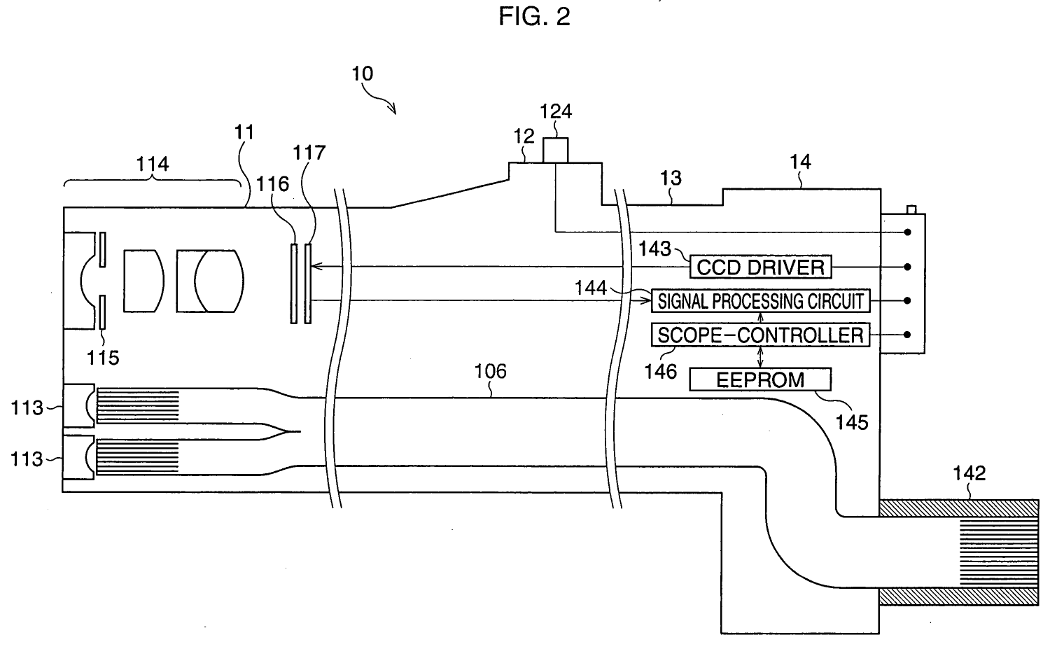

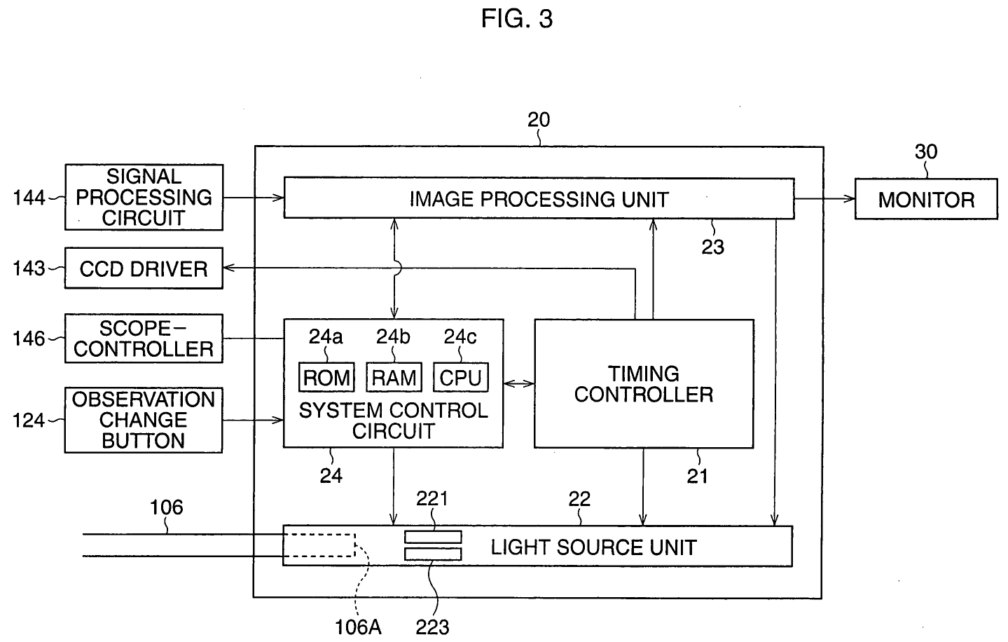

FIG. 2 is a block diagram of the video-scope 10. FIG. 3 is a block diagram of the video-processor 20.

As shown in FIG. 3, a light source unit 22 in the video-processor 20 has a first light source 221 such as xenon lamp, which emits white light, and a second light source 223 such as a semiconductor laser, which emits exciting-light. The spectrum distribution of the white light is generally uniform and spreads over the range of visible light. The exciting-light is light for emitting auto-fluorescent light from tissue of the observed portion, and has a narrow specific wavelength or spectrum. The light radiated from the first light source 221 or the second light source 223 enters into an incidence surface 106A of a light-guide 106.

As shown in FIG. 2, the light guide 106 of a fiber-optic bundle, which is provided in the video-scope 10, directs the light to the tip portion of the video-scope 10. The light from the light-guide 106 is emitted from the tip portion of the video-scope 10 via a diffusion lens 113, thus an observed subject is illuminated.

Light reflected on the subject passes through an optical system 114, an iris 115, and an exciting-light eliminating filter 116, so that the subject image is formed on a photo-sensor area of a CCD 117, which is provided in the tip portion of the video-scope 10. As described later, the exciting-light eliminating filter 116 blocks or cuts off the exciting-light.

In this embodiment, an on-chip color filter method using an on-chip color filer is applied. On the photo-sensor area of the CCD 117, a complementary color filer (not shown), checkered by four color elements, Yellow (Y), Magenta (Mg), Cyan (Cy), and Green (G), is arranged such that each area of the four color elements is opposite a pixel, and the pixels are two-dimensionally arranged in the photo-sensor area.

In the CCD 54, image-pixel signals, corresponding to light passing through the complementary color filter, are generated by photoelectric transform effect. A CCD driver 143 outputs clock pulse signals to the CCD 117 to read the image-pixel signals. The generated image-pixel signals are red from the CCD 117 at regular time intervals in accordance with the so called “color difference line sequential system”. Herein, the NTSC or PAL standard is applied as the video-standard, accordingly, one field worth of image-pixel signals is read from the CCD 117 at “ 1/60” or “ 1/50” of a second time intervals, and are then fed to an signal processing circuit 144.

In the signal processing circuit 144, predetermined processes are performed for the image-pixel signals so that luminance signals “Y” and color difference signals “Cb” and “Cr” are generated and then fed to an image processing-unit 23 in the video-processor 20.

A scope-controller 146 controls the video-scope 10, and outputs control signals to the signal processing circuit 144. In an EEPROM 145, data associated with the video-scope 10, such as an ID number, and further data associated with a signal process is stored as described later. The signal processing circuit 144 has a register (not shown) and the scope controller 146 writes data associated with the signal process in the register. The signal processing circuit 144 processes the image-pixel signals in accordance with the data stored in the register.

In the image processing unit 23 shown in FIG. 3, various processes, such as a white balance process and a gamma correction, are performed for the luminance and color difference signals “Y, Cb, Cr”, so that video signals are output to the monitor 30. Thus, an subject image is displayed on the monitor 30.

A timing controller 21 outputs clock pulse signals to each circuit in the video-process 20 and the CCD driver 143 in the video-scope 10 to synchronize the input and output of signals in the circuits. The system control circuit 24 including a ROM 24a, a RAM 24b, and a CPU 24C controls the video-processor 20, and outputs control signals to the light source unit 22.

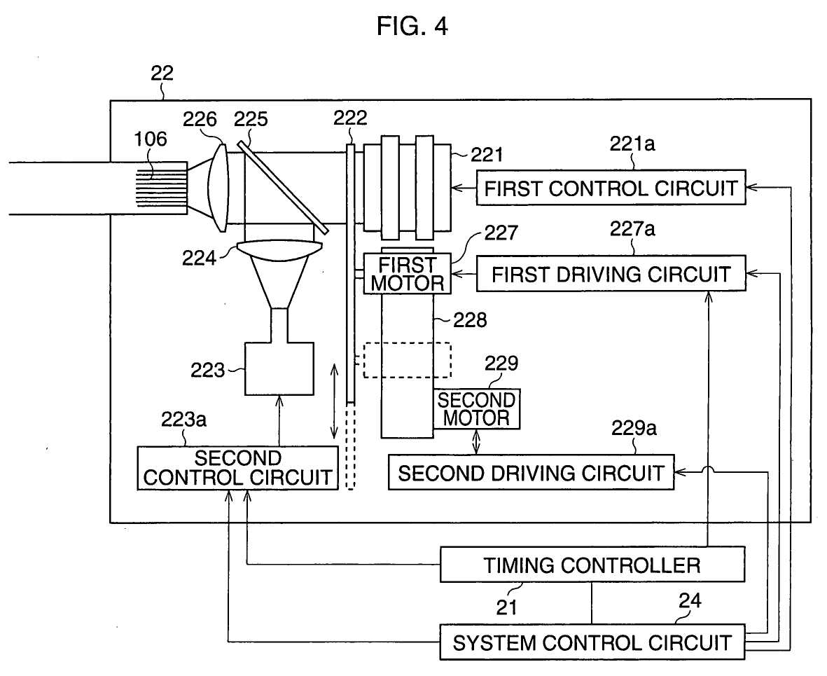

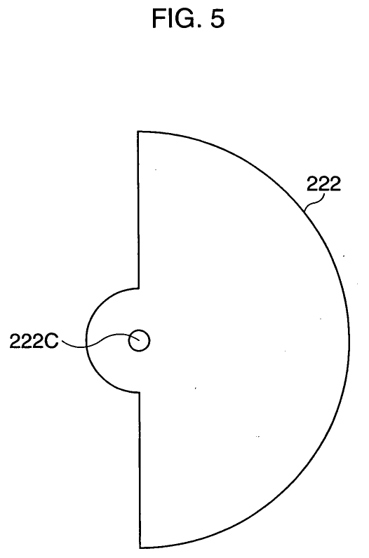

FIG. 4 is a block diagram of the light source unit 22. FIG. 5 is a front view of a rotary shutter.

As shown in FIG. 4, the light source unit 22 with the first light source 221 and the second light source 223 has a rotary shutter 222, a collimator lens 224, a dichroic mirror 225, and a collecting lens 226. The rotary shutter 222 is semicircle-shaped disk, as shown in FIG. 5, the center of the rotary shutter 222C is coaxially attached to a first motor 227. The rotary shutter 222 is arranged so as to cross a light-path of the light radiated from the first light source 221. While the rotary shutter 222 rotates by the rotation of the first motor 227, the parallel white light, emitted from the first light source 221, is periodically intercepted by the rotary shutter 222. The light passing through the dichroic mirror 225 and the collecting lends 226 enters into the light guide 106. The first control circuit 221a drives the first light source 221.

The second light source 223 of the semiconductor laser emits a laser beam having a given wavelength or spectrum. The second light source 223 selectively emits one laser beam having a particular narrow wavelength in a range of wavelengths corresponding to ultraviolet and visible rays. The second light source 223 has a plurality of laser diodes respectively emitting different spectrum light. The second control circuit 223a controls the outputting of the laser beams, namely, sets a laser beam to be emitted and drives a corresponding laser diode.

The collimator lens 224 collimates the light or laser beam radiated from the second light source 223, and the paralleled light is reflected on and the dichroic mirror 25, which is inclined by 45 degrees relative to the light-path of the first light source 221 and second light source 223. The light is directed to the light guide 226 along the light-path of the first light source 221.

A slider 228 slides so as to shift or reciprocate the first motor 227 and the rotary shutter 222 along a direction perpendicular to the light-path of the first light source 221. Thus, the rotary shutter 222 is selectively arranged to an interrupting-position for periodically blocking the light, and an outside-position. A second motor 229 is attached to the slider 222 via a pinion and gear mechanism (not shown). The slider 228 slides by the motion of the second motor 229. The first motor 227 and the second motor 229 are respectively driven by a first driving circuit 227a and a second driving circuit 229a. The system control circuit 24 controls the first and second driving circuits 227a and 229a, and the first and second control circuits 221a and 223a, in accordance with the selected observation mode.

When the normal observation mode is selected, the slider 228 slides to arrange the rotary shutter 222 at the outside-position, and the first control circuit 221 drives the first light source 221 so as to continuously radiate the white light. The second control circuit 223a does not drive the second light source 223. Thus, the white light continuously illuminates the subject.

When the auto-fluorescent observation mode is selected, the second control circuit 223a drives a given laser diode in the second light source 223 so as to continuously radiates the exciting-light. The first control circuit 221a does not drive the first light source 221. Thus, the exciting-light illuminates the subject.

When the special observation mode is selected, the slider 228 slides so as to arrange the rotary shutter 222 in the interrupting-position, and the first control circuit 221a drives the light source 221. The first driving circuit 227a controls the rotation of the first motor 227 such that the semicircular-shaped rotary shutter 222 rotates by one-rotation between the first (odd) field interval ( 1/60 or 1/50 seconds). On the other hand, the second control circuit 223a controls the second light source 223 such that the exciting-light periodically is emitted only for the second (even) field interval. Thus, the white light and the exciting-light alternately illuminate the subject.

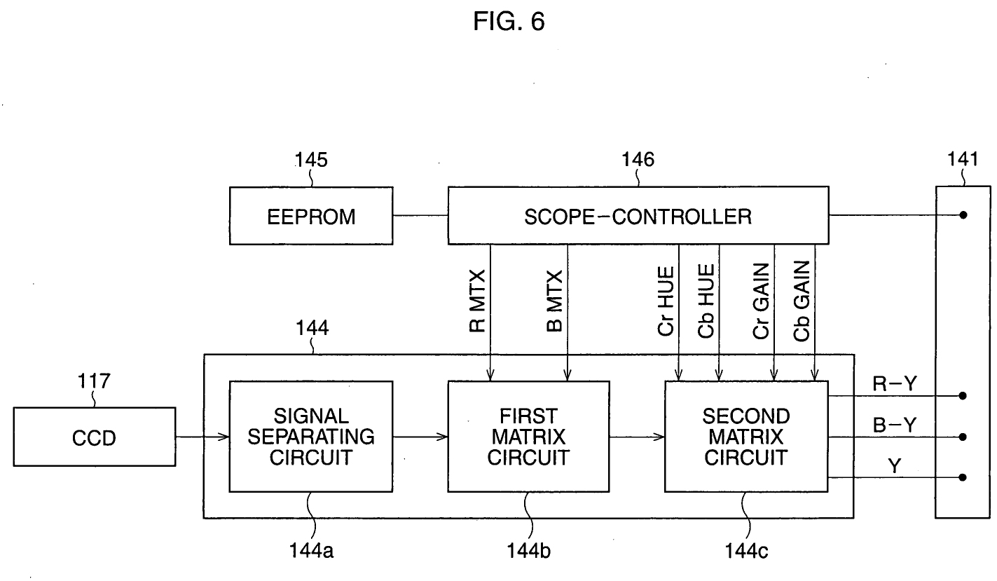

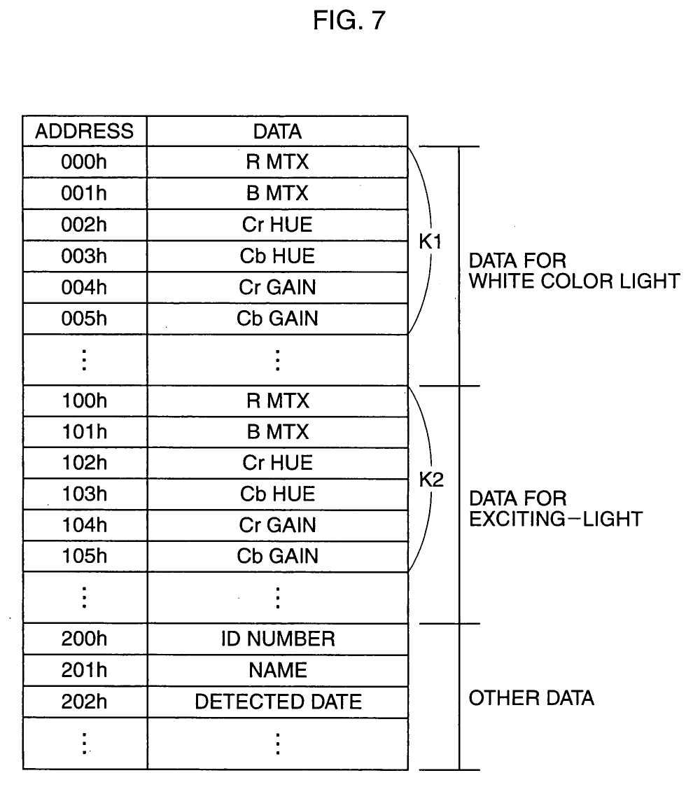

FIG. 6 is a block diagram of the video-scope 10. FIG. 7 is a view showing data associated with the signal process.

The signal processing circuit 144 has a signal separating circuit 144a, a first matrix circuit 144b, and a second matrix circuit 144c. In the signal separating circuit 144a, the complementary color signals (Mg+Ye, G+Cy, G+Ye, Mg+Cy) are separated into initial luminance signals “Ya” and initial chrominance signal “C′”, which are fed to the first matrix circuit 144b. The initial luminance signals Ya (=2R+3G+2B) are signals corresponding to the luminance signals “Y”. On the other hand, the initial chrominance signals C′ include initial color difference signals “C′r” (=2R−G) and c′b (2B−G), which respectively correspond to color difference signals Cr (=R−Y) and color difference signals Cb (=B−Y).

In the first matrix circuit 144b, primary color signals composed of “Red (R), Green (G), and blue (B)” signal components, are generated by the following formula, on the basis of the initial luminance signals Ya and the initial chrominance signals C′. Note that, coefficients “α” and “β” respectively indicate values of data, which are designated as “R MATX” and “B MITX” in FIG. 7. Data of the coefficients “R MATX” and “B MATX” are fed from the scope-controller 146 to the matrix circuit 144b.
R=C′r+α×Ya (1)
B=−C′b+β×(Ya−C′r) (2)
G=Ya−C′r+C′b (3)
The generated primary color signals “R, G, and B” are fed to the second matrix circuit 144c.

In the second matrix circuit 144c, luminance signals Y and color difference signals Cb (=B−Y) and Cr (=R−Y) are generated from the primary color signals by using matrix coefficients. Then, the color difference signals Cb and Cr, constructing chrominance signals, are subjected to a phase adjustment in accordance with phase control data “Cb HUE” and “Cr HUE”. Further, the output level of the color difference signals Cb and Cr are adjusted in accordance with the output level adjusting data “Cb GAIN” and “Cr GAIN”. These coefficients are fed from the scope-controller 146. The generated luminance signals Y and the color difference signals Cb and Cr are fed to the processor 20 via a connector 141 of the video-scope 10.

The data associated with the signal process, shown in FIG. 7, is stored in the EEPROM 145 in advance, at given addresses. In the present embodiment, a set of data for white light “K1”, and a set of data for exciting-light “K2” are prepared and stored. The set of data for white light “K1” is utilized for processing image-pixel signals obtained by the white light, whereas the set of data for exciting-light “K2” is utilized for processing image-pixel signals obtained by the exciting-light.

When the normal observation is selected, the set of data for white light “K1” is read from the EEPROM 145 and is then written at given addresses of the register provided in the signal processing circuit 144. The signal processing circuit 144 processes the initial luminance signals “Ya” and the initial chrominance signals “C′”, in accordance with the set of data “K1”.

As described above, the wavelength-band of the exciting-light is various, and light having a particular wavelength-band can be selectively radiated as an exciting-light. For example, one wavelength-band of the exciting-light is in the wavelength range of ultraviolet light (approximately 400 nm), or in the wavelength range between the ultraviolet light and blue light (400 to 480 nm), or another range. In the present embodiment, a plurality of video-scopes, which is available for the auto-fluorescent observation, is selectively connected to the video-processor 20, and each video-scope has a different light-eliminating filter with respect to the spectrum transmitting characteristics. As described later, the second light source 223 radiates exciting-light corresponding to the light-eliminating filter in a connected video-scope. Namely, the second light source 223 radiates exciting-light blocked in accordance with the spectrum transmittance characteristics of the filter 116.

When the exciting-light is included in the range of the visible light, namely, the light eliminating filter 116 blocks a part of the visible light, which is the same as the exciting light, a luminance and a color in the observed image changes in the normal observation. The set of data “K1” is data that compensates for the change of signal components due to light cut-off by the eliminating filter 116. Namely, the image-pixel signals are corrected by the set of data “K1” so as not to change the luminance and color in the observed image. The set of data “K1” stored in the EEPROM 145 is predetermined in accordance with the spectrum transmitting characteristics of the light-eliminating filter 116, the values of the data “K1” is defined in each connectable video-scope. When the wavelength-band of the exciting light is outside that of visible light, the set of data “K1” is the same as that of the video-scope that is exclusive for the normal observation, without a light eliminating filter.

When the auto-fluorescent observation is selected, the set of data for auto-fluorescent light “K2” is written on given addresses of the register. The wavelength band of the auto-fluorescent light radiated from the tissue is included in that of the visible light, which causes the change of the luminance and color in the observed image obtained by the auto-fluorescent light. The set of data “K2”, similarly to the set of data “K1”, compensates for the change of signal components due to the blocked light.

When the special observation mode is selected, the set of data “K1” and the set of data “K2” are written in the register. The scope-controller 146 controls the signal processing circuit 144 so as to process the image pixel signals obtained by the white light on the basis of the set of data “K1”, and process the image-pixel signals obtained by the fluorescent on the basis of the set of data “K2”, namely, processes the image-pixel signals by alternately use the data “K1” and “K2” at one-field intervals.

In the image processing unit 23 provided in the video-processor 20, the analog luminance signals Y and the color difference signals Cb, Cr, which are output from the video-scope 10, are transformed to digital R, G, B image signals corresponding to a given color management system such as the s-RGB Color Space. When the normal observation or the auto-fluorescent observation is selected, the digital R,G,B image signals are temporarily stored in a memory (not shown) provided in the image processing unit 23 at field time intervals, and are then transformed to video signals such as NTSC signals, which are output to the monitor 30. On the other hand, when the special observation mode is selected, luminance difference image signals, which indicate a luminance difference between R, G, B image signals obtained by the white light and R, G, B image signals obtained by the fluorescent light, are generated. Then, one field worth of R, G, B image signals and the luminance difference image signals are synthesized and are output to the monitor 30 as video signals. Thus, a composite observed image is displayed on the monitor 30.

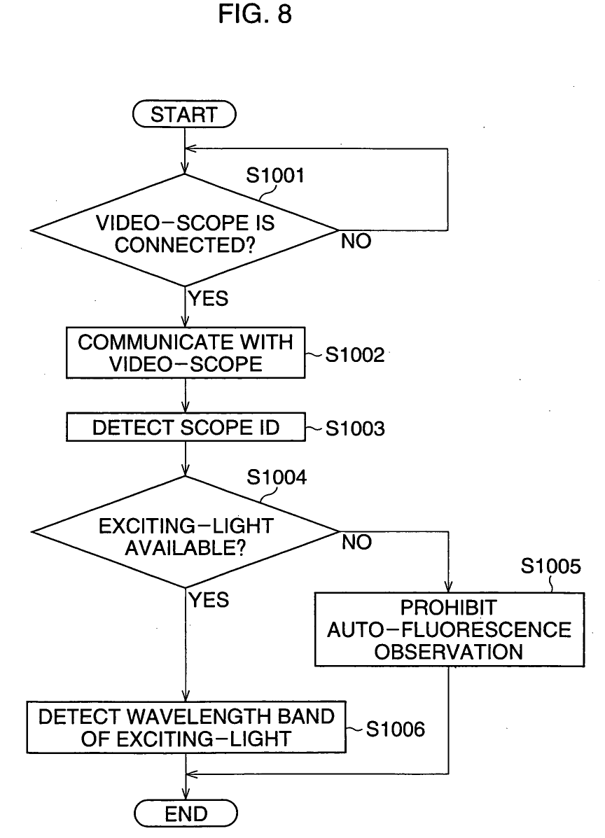

FIG. 8 is a flowchart of the initial setting process performed by the system control circuit 24. When electric power is turned ON, or the video-scope is detached from the video-processor 20, the process is started.

In Step S1001, it is determined whether the video-scope 10 is connected to the video-processor 20. When it is determined that the video-scope is not connected to the video-processor 20, Step S1001 is repeatedly performed. On the other hand, when it is determined that that the video-scope is connected to the video-processor 20, the process goes to Step S1002, wherein the data associated with the connected video-scope is transmitted from the video-scope 10. In Step S1003, the ID number of the video-scope 10 is detected.

In Step S1004, it is determined whether the connected video-scope 10 is adaptable to the auto-fluorescent observation, in other words, it is determined whether the video-scope has an exciting-light eliminating filter. In the ROM 24a, a table, indicating the relationship between the ID number of the video-scope and the possibility of the auto-fluorescent observation, is stored as data. When it is determined that the connected video-scope 10 is adaptable to the auto-fluorescent observation, the process goes to Step S1006, wherein the wavelength band or a spectrum of the exciting-light is detected. On the other hand, when it is determined that the connected video-scope 10 is not adaptable to the auto-fluorescent observation, namely, the connected video-scope is used for only the normal observation, the process goes to Step S1005, wherein the second control circuit 223a is set so as not to activate the second light source 223 in the case of the auto-fluorescent observation.

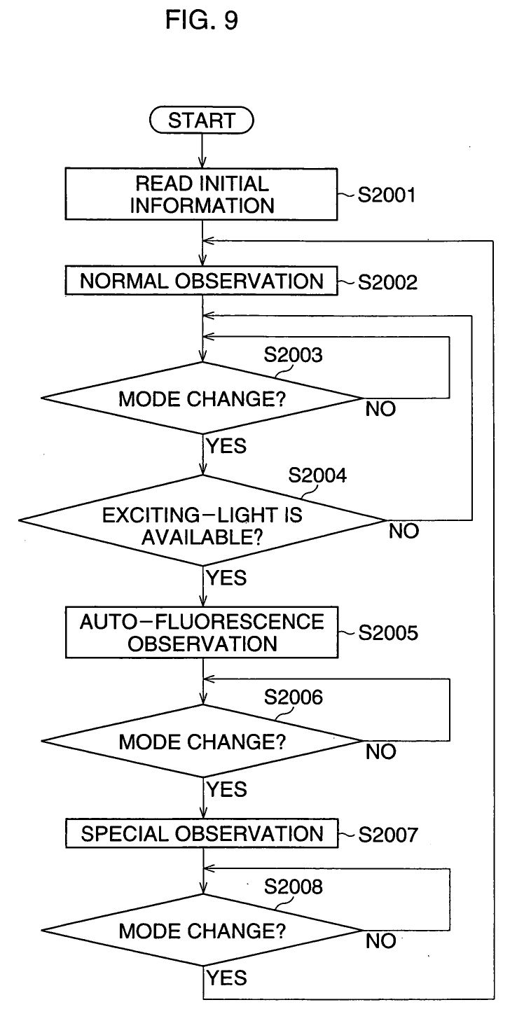

FIG. 9 is a flowchart of the main routine performed by the system control circuit 24. The process is started after the initial setting process shown in FIG. 8 is terminated.

In Step S2001, initial information is read from the ROM 24a. In Step S2002, the system control circuit 24 outputs control signals to the light source unit 22 and the scope-controller 146 so as to practice the normal observation.

In Step S2003, it is determined whether the observation mode has been changed by operating the observation change button 124. When it is determined that the observation mode has not been changed, Step S2003 is repeatedly performed. On the other hand, when it is determined that the observation mode has been changed, the process goes to Step S2004.

In Step S2004, it is determined whether the connected video-scope is adaptable to the auto-fluorescent observation. When it is determined that the connected video-scope is not adaptable to the auto-fluorescent observation, the process returns to Step S2003, and the normal observation is maintained. On the other hand, when it is determined that the connected video-scope is adaptable to the auto-fluorescent observation, the process goes to Step S2005, wherein the system control circuit 24 outputs control signals to the light source unit 22 and the scope-controller 146 so as to enable the auto-fluorescent observation.

In Step S2006, it is determined whether the observation mode has been changed by operating the observation change button 124. When it is determined that the observation mode has not been changed, Step S2006 is repeatedly performed. On the other hand, when it is determined that the observation mode has been changed, the process goes to Step S2007. In Step S2007, the system control circuit 24 outputs control signals to the light source unit 22 and the scope-controller 146 so as to enable the special observation. In Step S2008, it is determined whether the observation mode has been changed by operating the observation change button 124. When it is determined that the observation mode has not been changed, Step S2008 is repeatedly performed. On the other hand, when it is determined that the observation mode has been changed, the process returns to Step S2002.

In this way, in the present embodiment, the video-scope 10 with the exciting-light eliminating filter 116 is connected to the video-processor 22 with the first light source 221 emitting the white light and the second light source 223 emitting the exciting-light. In the normal observation, the signal processing circuit 144 processes the image signals in accordance with the set of data “K1” representing the coefficients, which compensate for the image signal, such that a luminance and/or color in the observed image is not changed due to light cut off by the exciting-light eliminating filter 116. On the other hand, in the case of the auto-fluorescent observation, the signal processing circuit 144 processes the image signals in accordance with the set of data “K2”. In the case of the special observation, the image signals are processed by alternately using the set of data “K1” and the set of data “K2”. Consequently, a composite observed image with adequate luminance and color is displayed on the monitor 30.

The signal process using the series of coefficients may be performed in the video-processor instead of the video-scope. In this case, the video-processor detects the type of video-scope, namely, the spectrum transmittance characteristics, and selects the corresponding coefficient data, which is stored in a memory in advance. The signal processing circuit may process the white balance data or gamma data by using coefficients for compensation. The image processing circuit may compensate for only luminance or color. Any signal process without coefficients for compensation may be performed. A video-scope exclusive for the auto-fluorescent observation may be connected to the video-processor.

Finally, it will be understood by those skilled in the art that the foregoing description is of preferred embodiments of the device, and that various changes and modifications may be made to the present invention without departing from the spirit and scope thereof.

The present disclosure relates to subject matters contained in Japanese Patent Application No. 2005-030686 (filed on Feb. 7, 2005), which is expressly incorporated herein, by reference, in its entirety.

1. An electronic endoscope system comprising:
a light source unit that is capable of selectively emitting normal-light and exciting-light;
a video-scope with an image sensor, that has an exciting-light eliminating filter that is provided in front of said image sensor and that blocks the exciting-light; and
an image signal processor that processes image signals read from said image sensor, to generates video signals,
wherein said image signal processor processes the image signals so as to compensate for a change of at least one of luminance and color in an observed image that occurs due to light blocked in accordance with spectrum transmittance characteristics of said exciting-light eliminating filter.

2. The electronic endoscope system of claim 1, wherein said image signal processor comprises:
a signal processing circuit that processes the image signals by using coefficients; and
a signal controller that sets the coefficients to compensation coefficients that compensate for the change.

3. The electronic endoscope system of claim 2, wherein said signal processing circuit generates luminance signals and color difference signals by a matrix-operation using the coefficients.

4. The electronic endoscope system of claim 2, wherein the signal controller sets normal-light coefficients corresponding to the normal-light as the compensation coefficients,
wherein said signal processing circuit processes the image signals by using the normal-light coefficients when said light source unit emits the normal-light.

5. The electronic endoscope system of claim 2, wherein the signal controller sets fluorescent coefficients corresponding to the fluorescent light as the compensation coefficients,
wherein said signal processing circuit processes the image signals by using the fluorescent coefficients when said light source unit emits the exciting-light.

6. The electronic endoscope of claim 2, further comprising:
a first memory that stores the compensation coefficients as data,
a second memory that is provided in said signal processing circuit and that stores the compensation coefficients as data,
wherein said signal controller reads the compensation coefficients from said first memory and writes the compensation coefficients in said second memory, said signal processing circuit processing the image signals in accordance with the written compensation coefficients.

7. The electronic endoscope of claim 1, wherein said light source unit alternately emits the normal-light and the exciting-light while synchronizing with signal-reading time-intervals from said image sensor,
wherein said image signal processor generates composite image signals by synthesizing normal-light image signals obtained by the normal-light and fluorescent image signals obtained by the exciting-light.

8. The electronic endoscope system of claim 1, wherein said image signal processor is provided in said video-scope.

9. The electronic endoscope system of claim 1, further comprising:
a video-scope type detector that detects whether a connected video-scope is adaptable to the exciting-light; and
a light source controller that prevents said light source unit from emitting the exciting-light when the connected video-scope is not adaptable to the exciting-light.

10. The electronic endoscope system of claim 1, further comprising:
a video-scope type detector that detects the spectrum transmittance characteristics of the exciting-light eliminating filter provided in a connected video-scope,
wherein said light source unit emits exciting-light depending upon the spectrum transmittance characteristics.

11. A video-scope with an image sensor for an electronic endoscope system comprising:
an exciting-light eliminating filter that is provided in front of said image sensor and that blocks the exciting-light; and
an image signal processor that processes image signals read from said image sensor, to generate video signals,
wherein said image signal processor processes the image signals so as to compensate for a change of at least one of luminance and color in an observed image that occurs due to light blocked in accordance with spectrum transmittance characteristics of said exciting-light eliminating filter.

12. A video-processor for an electronic endoscope system, the video scope according to claim 11 is connected to the video-processor, said video-processor comprising:
a light source unit that is capable of selectively emitting normal-light and exciting-light; and
a video-scope type detector that detects the spectrum transmittance characteristics of the exciting-light eliminating filter provided in a connected video-scope,
wherein said light source unit emits exciting-light depending upon the spectrum transmittance characteristics.

13. An apparatus for processing image signals obtained by a video-scope with an image sensor, said video-scope comprising an exciting-light eliminating filter that is provided in front of said image sensor and that blocks the exciting-light, said apparatus comprising:
a signal processing circuit that processes image signals by using coefficients to generate video signals; and
a signal controller that sets the coefficients to compensation coefficients that compensate for a change of at least one of luminance and color in an observed image that occurs due to light blocked in accordance with the spectrum transmittance characteristics of said exciting-light eliminating filter.

14. A method for processing image signals obtained by a video-scope with an image sensor, said video-scope comprising an exciting-light eliminating filter that is provided in the front of said image sensor and that blocks the exciting-light, said method comprising:
processing image signals by using coefficients to generate video signals; and
setting the coefficients to compensation coefficients that compensate for a change of at least one of luminance and color in an observed image that occurs due to light blocked in accordance with the spectrum transmittance characteristics of said exciting-light eliminating filter.
